(19) United States
(12) Patent Application Publication
(10) Pub. No.: US 2026/0260745 A1
(43) Pub. Date: Sep. 3, 2026

(54) A Prediction Algorithm for Determining the Optimized Cost-Effectiveness of Advanced Tumors Treatment

(71) Applicants: Shekoufeh Nikfar, Tehran (IR); Marzieh Nosrati, Tehran (IR)

(72) Inventors: Marzieh Nosrati, Tehran (IR); Shekoufeh Nikfar, Tehran (IR); Mohammad Abdollahi, Tehran (IR); Mohammadhossein Sharifnia, Tehran (IR); Mahdi Asfia, Tehran (IR)

(21) Appl. No.: 18/713,650

(22) PCT Filed: Dec. 6, 2022
(86) PCT No.: PCT/IB2022/061845

§ 371 Date: May 26, 2024

(51) Int. Cl. G16H 50/20; G16H 10/20; G16H 50/30; G16H 50/70

(52) U.S. Cl. CPC G16H 50/20; G16H 10/20; G16H 50/30; G16H 50/70

(57) ABSTRACT

One of the most critical problems in the area of therapeutic interventions for cancer patients has been the choice of the therapeutic regimen that has been ineffective in the long-term complications in the patient's treatment path. On the one hand, this matter has led to the disease progression and declined the patients' quality of life because of lacking sufficient effectiveness, and on the other hand, it has caused numerous other problems in the patient's quality of life due to the extensive range of complications, particularly in the long-term use of the relevant regimen. Information and communication technology (ICT) allocated in treatment plans and paths related to health promotion, including therapeutic guidance or patient monitoring compliance associated with drug regimens and clinical interventions, as well as ICT assigned in the paths of diagnosis, simulation, and data mining of medical studies, for the purpose of health assessments and individual risks, are among the objectives of the invention. To improve the listed problems, the claimed invention investigated the methods and prediction algorithms based on cost-effectiveness parameters using precision medicine reference models for optimizing the cost and subsequently, achieving the highest level of effectiveness in patients with cancers, especially patients involved with a more advanced type of cancer characterized by mismatch repair deficiency.

### FIELD OF THE INVENTION

The background of the invention is in the fields of developing a multivariable cost-effectiveness prediction algorithm based on precision medicine elements and Kaplan-Meier survival analysis, information and communication technology specialized in the therapeutic plans and approaches related to health promotion; including, therapeutic guidance or comprehensive monitoring of patients related to drug regimens and clinical interventions. Also, this invention covers the area of disease diagnosis and prediction pathways, slope creation, and data mining of medical studies, for health assessments and individual risks. This invention's field can be searched with International patent codes (IPC) G16H20/10, G16H50/00, and G16H50/30 in various patent databases.

### BACKGROUND OF THE INVENTION

The U.S. Pat. No. 10,282,512B2 patent with the title of “Clinical decision-making artificial intelligence object-oriented system and method” involves a system and method of providing decision support for assisting medical treatment decision-making. A patient agent software module processes information about a particular patient. A doctor agent software module processes information about the health status of a particular patient, beliefs relating to patient treatments, and the actual effects of treatment decisions. By filtering information over time from the patient agent into the doctor agent, a plurality of decision-outcome nodes is created and formed into a patient-specific outcome tree with the plurality of decision-outcome nodes.

An optimal treatment is determined by evaluating the plurality of decision-outcome nodes with a cost-per-unit change function to output the optimal treatment. When additional information is available from at least one of the patient agents and the doctor agent, the filtering, creating, and determining steps are repeated thus allowing for the system to “reason over time”, continuously updating and learning as new information is received. From the methods and patterns based on artificial intelligence based on the patient's conditions and clinical manifestations, the electronic data recorded by the connected equipment of the patients selects the most effective treatment approach from the point of view of effectiveness.

The U.S. Pat. No. 7,890,267B2 patent with the title of “Prognostic and diagnostic method for cancer therapy” provides novel methods and kits for diagnosing the presence of cancer within a patient, and for determining whether a subject who has cancer is susceptible to different types of treatment regimens. The cancers to be tested include, but are not limited to, prostate, breast, lung, gastric, ovarian, bladder, lymphoma, mesothelioma, medulloblastoma, glioma, and AML. Identification of therapy-resistant patients early in their treatment regimen can lead to a change in therapy in order to achieve a more successful outcome.

One embodiment of the present invention is directed to a method for diagnosing cancer or predicting cancer-therapy outcomes by detecting the expression levels of multiple markers in the same cell at the same time, and scoring their expression as being above a certain threshold, wherein the markers are from a particular pathway related to cancer, with the score being indicative or a cancer diagnosis or a prognosis for cancer-therapy failure. This method can be used to diagnose cancer or predict cancer therapy outcomes for a variety of cancers. The markers can come from any pathway involved in the regulation of cancer, including specifically the PcG pathway and the “stemness” pathway. The markers can be mRNA, microRNA, DNA, or protein. Statistical models and the use of the survival model in a specific path and treatment regimen, compare the prognosis of patients in recovery with the selected treatment path by referring to different gene expression states.

The U.S. Pat. No. 10,047,403B2 patent with the title of “Diagnostic methods for determining prognosis of non-small cell lung cancer disclosed methods for identifying early stage non-small-cell lung cancer (NSCLC) patients who will have an unfavorable prognosis for the recurrence of lung cancer after surgical resection. The methods are based in part on the discovery of chromosomal copy number abnormalities that can be used for prognostic classification. The methods preferably use fluorescence in situ hybridization with fluorescently labeled nucleic acid probes to hybridize to patient samples to quantify the chromosomal copy number of these genetic loci. From the survival model, it compares the prognosis of patients with this type of cancer in a specific treatment regimen based on the predominance of a specific marker of the causative gene in the same period of treatment, in order to relate the effectiveness of the treatment regimen with selected specific markers.

### SUMMARY OF THE INVENTION

A multivariable cost-effectiveness prediction algorithm is employed for the choice of the type of interventions, therapeutic approach, and therapeutic regimen in severe, chronic, and life-threatening diseases, such as cancer, designed with a precision medicine approach. this algorithm applies to advanced diseases with progressive, life-threatening characteristics, limited statistical population, heavy, expensive, chronic therapeutic regimens with low effectiveness, and low probability of reaching the complete response phase (full recovery), such as malignancies and cancerous tumors. This algorithm is composed of two general parts: effectiveness function and treatment cost function. In the case of a small statistical population in the clinical trials conducted to check the effectiveness of the target drug with a precision medicine approach, to increase the population, integrating and expanding the results and clinical data of various trials carried out by the claimed drug, in different cancers, with the commonality in connection with the presence of the claimed biomarker and subsequently, the similarity in the amount and effect of this claimed variable in the effectiveness of the treatment will be employed.

### BRIEF DESCRIPTION OF DRAWINGS

FIG. 1: Represents the analytical diagram of patients' survival with the Kaplan-Meier model in the Overall Survival (OS) mode to compare the Pembrolizumab monoclonal antibody therapeutic regimen (upper axis) and the comparison arm of the standard therapeutic regimen (lower axis) in patients experienced with endometrial cancer with MSI-H/dMMR biomarker (Microsatellite instability-high (MSI-H) and deficient mismatch repair (dMMR). The y-axis shows cum survival parameter, and the x-axis indicates the time parameter.

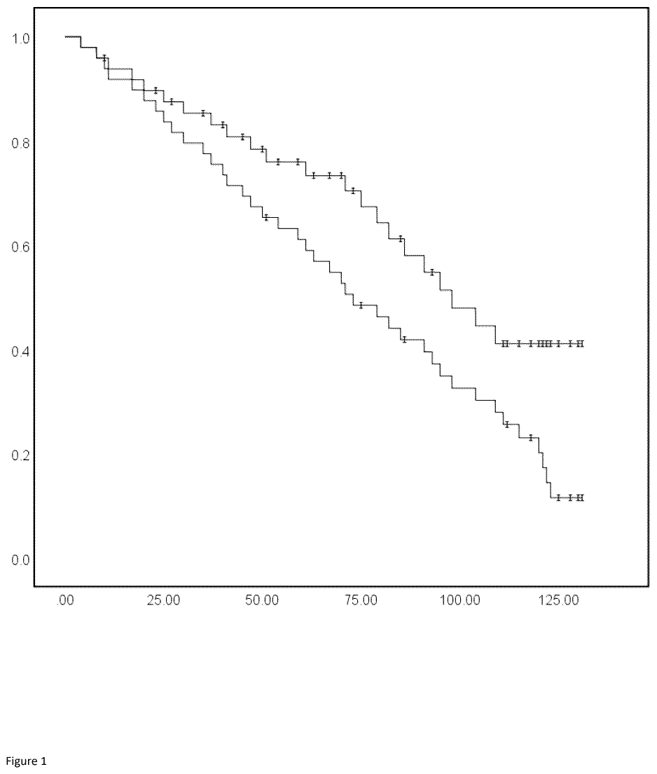

FIG. 2: Displays the analytical diagram of patients' survival with the Kaplan-Meier model in the Progression-Free Survival (PFS) mode to compare the Pembrolizumab monoclonal antibody therapeutic regimen (upper axis) and the comparison arm of the standard therapeutic regimen (lower axis) in patients experienced with endometrial cancer with MSI-H/dMMR biomarker. The y-axis shows cum survival parameter, and the x-axis indicates the time parameter.

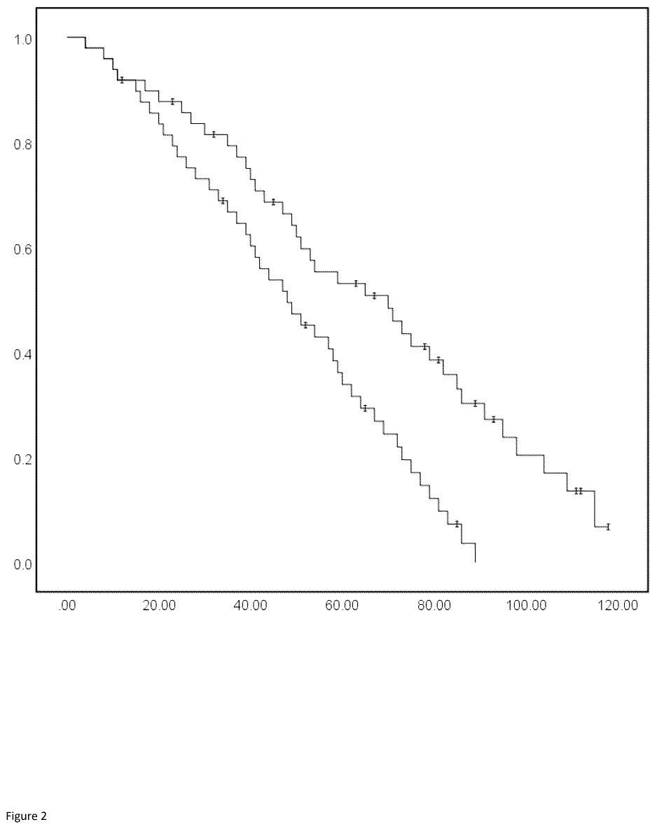

FIG. 3: Depicts the analytical diagram of patients' survival with the Hazard analysis approach with the Kaplan-Meier model in Complete-Response Survival (CRS) mode to compare the Pembrolizumab monoclonal antibody therapeutic regimen (upper axis) and the comparison arm of the standard therapeutic regimen (lower axis) in patients experienced with endometrial cancer with MSI-H/dMMR biomarker. The y-axis shows cum survival parameter, and the x-axis indicates the time parameter.

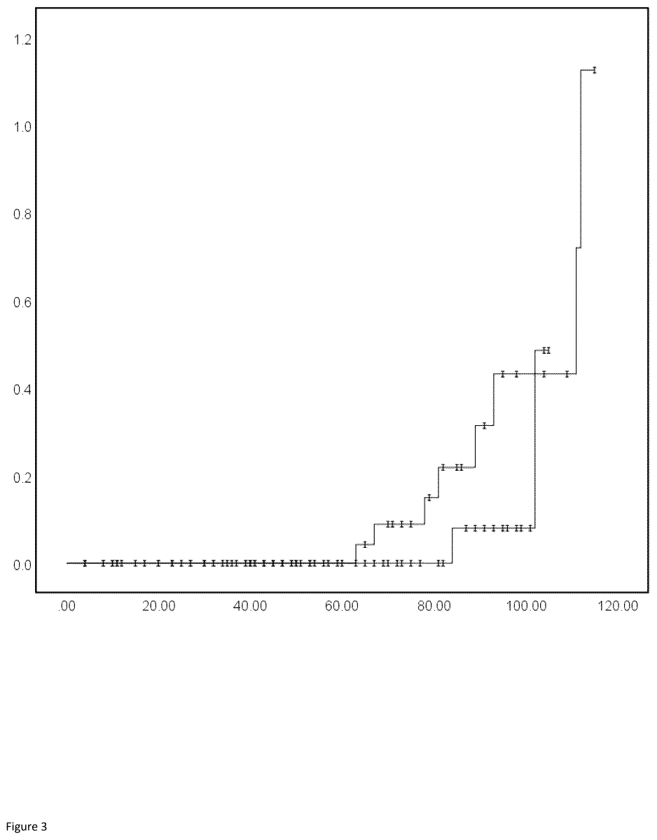

FIG. 4: Illustrates the analytical diagram of patients' survival with the Kaplan-Meier model in the Overall Survival (OS) mode to compare the Pembrolizumab monoclonal antibody therapeutic regimen (upper axis) and the comparison arm of the standard therapeutic regimen (lower axis) in patients experienced with stomach cancer with MSI-H/dMMR biomarker. The y-axis shows cum survival parameter, and the x-axis indicates the time parameter.

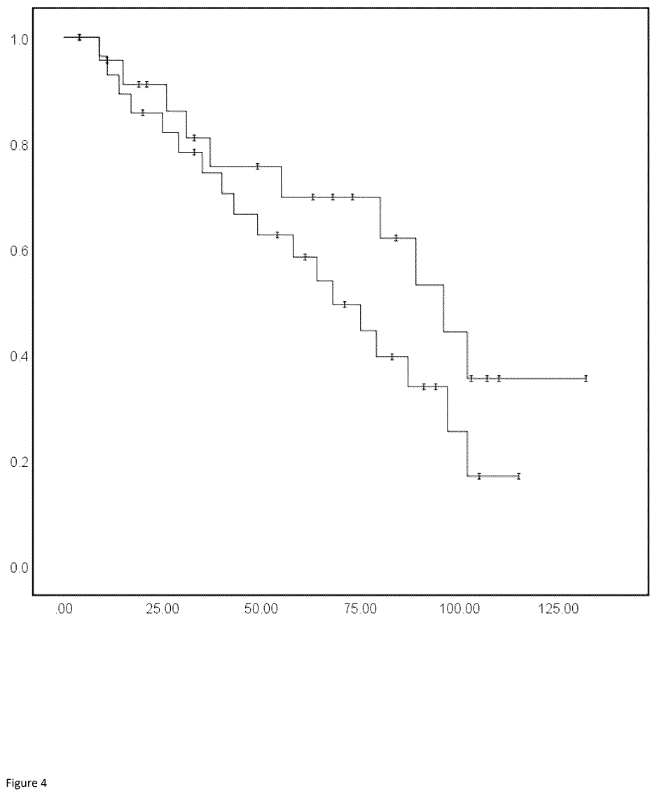

FIG. 5: Demonstrates the analytical diagram of patients' survival with the Kaplan-Meier model in the Progression-Free Survival (PFS) mode to compare the Pembrolizumab monoclonal antibody therapeutic regimen (upper axis) and the comparison arm of the standard therapeutic regimen (lower axis) in patients experienced with stomach cancer with MSI-H/dMMR biomarker. The y-axis shows cum survival parameter, and the x-axis indicates the time parameter.

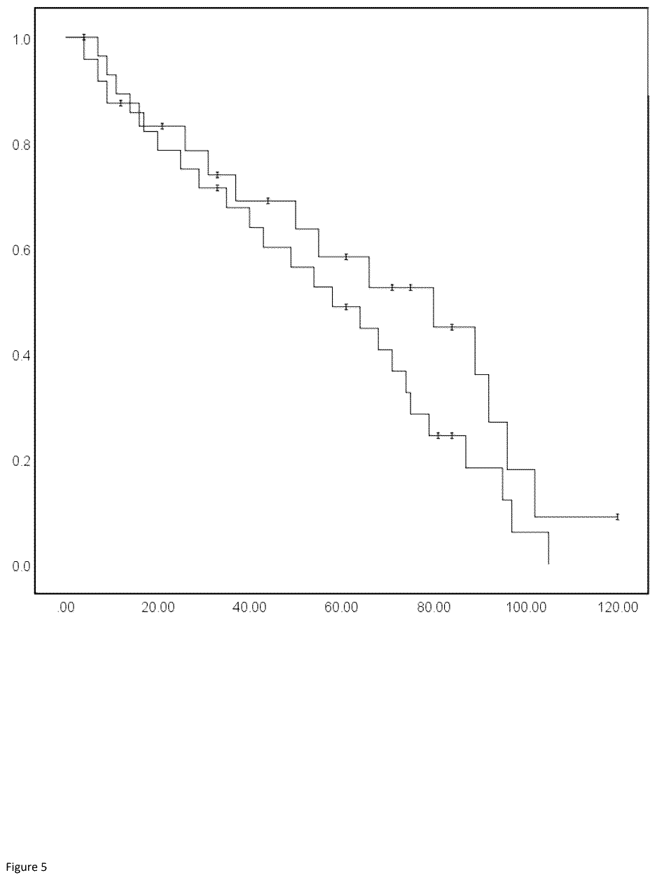

FIG. 6: Represents the analytical diagram of patients' survival with the Hazard analysis approach with the Kaplan-Meier model in Complete-Response Survival (CRS) mode to compare the Pembrolizumab monoclonal antibody therapeutic regimen (upper axis) and the comparison arm of the standard therapeutic regimen (lower axis) in patients experienced with stomach cancer with MSI-H/dMMR biomarker. The y-axis shows cum survival parameter, and the x-axis indicates the time parameter.

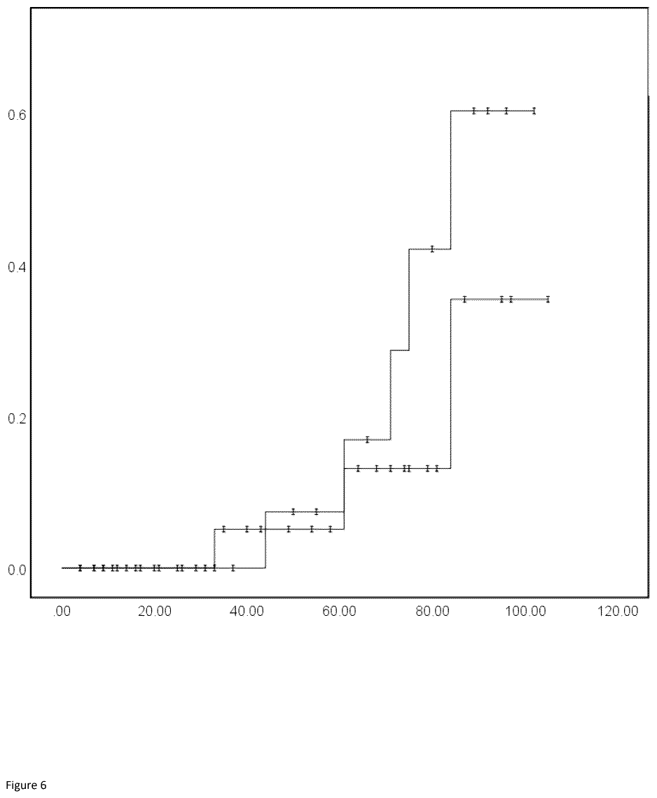

FIG. 7: Displays the analytical diagram of patients' survival with the Kaplan-Meier model in the Overall Survival (OS) mode to compare the Pembrolizumab monoclonal antibody therapeutic regimen (upper axis) and the comparison arm of the standard therapeutic regimen (lower axis) in patients experienced with pancreatic cancer with MSI-H/dMMR biomarker. The y-axis shows cum survival parameter, and the x-axis indicates the time parameter.

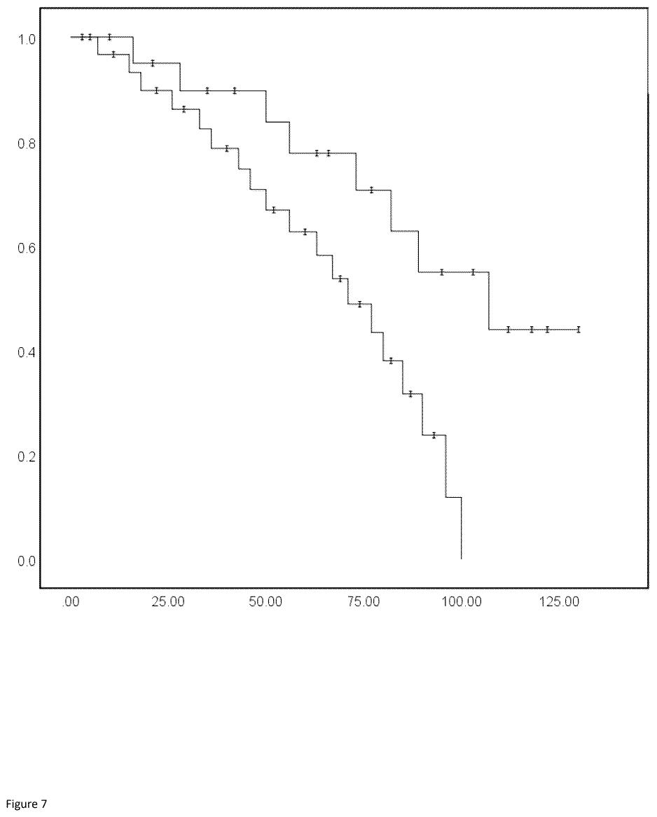

FIG. 8: Depicts the analytical diagram of patients' survival with the Kaplan-Meier model in the Progression-Free Survival (PFS) mode to compare the Pembrolizumab monoclonal antibody therapeutic regimen (upper axis) and the comparison arm of the standard therapeutic regimen (lower axis) in patients experienced with pancreatic cancer with MSI-H/dMMR biomarker. The y-axis shows cum survival parameter, and the x-axis indicates the time parameter.

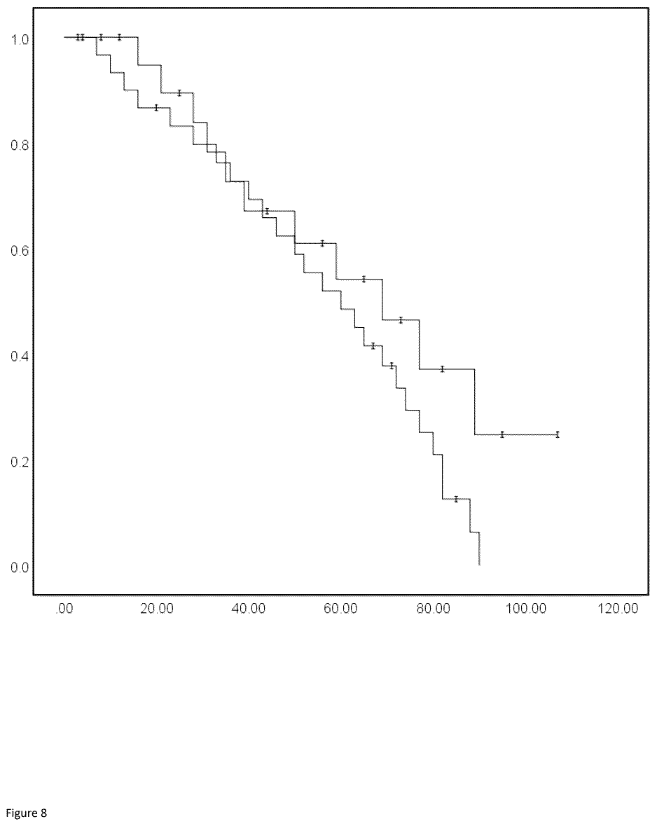

FIG. 9: Illustrates the analytical diagram of patients' survival with the Hazard analysis approach with the Kaplan-Meier model in Complete-Response Survival (CRS) mode to compare the Pembrolizumab monoclonal antibody therapeutic regimen (upper axis) and the comparison arm of the standard therapeutic regimen (lower axis) in patients experienced with pancreatic cancer with MSI-H/dMMR biomarker. The y-axis shows cum survival parameter, and the x-axis indicates the time parameter.

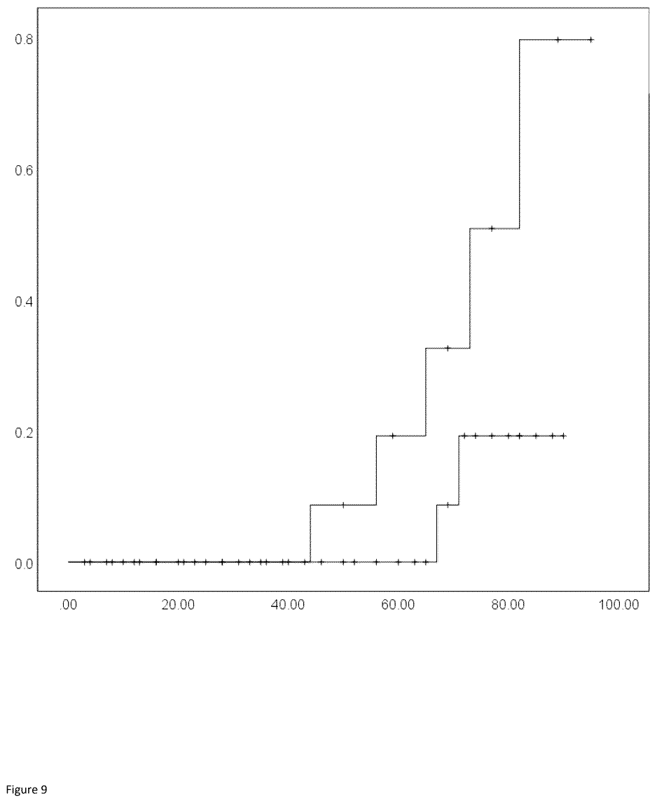

### DETAILED DESCRIPTION OF THE INVENTION

Maximum upgrading of the health level of every society is among the fundamental elements and prospects of the government structure. Thus, policymakers and decision-makers engaged in this area are obligated to offer novel techniques and patterns based on Information and Communication Technologies (ICT), resulting in the maximization of this issue and subsequently managing the limited budget under their control in the sphere of medical care and treatment optimally. Hence, making decisions in connection with the resource allocation for intervention has an opportunity cost. This means forgone health, which could have taken place following the allocation of the same resource to another intervention. One of the newfound and innovative interventions in the health field is the precision medicine approach, sometimes known as “personalized medicine”.

The application of this approach in the domain of pharmacotherapy means utilizing clinical information and genetic algorithms of patients for the choice of the right medicine with the appropriate treatment dose, which causes an improvement in outcomes and a decline in the side effects of treatment. However, the high price of drugs with a precision medicine approach has decreased access to treatment. Taking into account the peculiar features of the personalized medicine approach, carrying out various clinical evaluation studies and the challenges ahead in economic evaluation studies have prompted experts in the field of health economics and outcome research to look for generating proper methods and models to model the economic consequences and costs of the precision medicine strategy. Because of the high price of these drugs, using conventional economic evaluation models leads to incremental costs beyond the threshold of payment approved by a lot of countries, particularly developing countries. Concerning the special characteristics of the precision medicine approach, which focuses on the existence of a specific gene map rather than a mere examination of a disease's clinical presentations and causes a change in the type of clinical studies of these drugs as well, performing economic evaluation studies with the classical approach is questioned. In this respect, the claimed invention investigated the methods and algorithms based on cost-effectiveness parameters using precision medicine reference models for optimizing the cost and subsequently, achieving the highest level of effectiveness in patients with cancers, especially patients involved with a more advanced type of cancer characterized by mismatch repair deficiency.

In the claimed invention, the cost-effectiveness optimization algorithms of treatment methods used in precision medicine were exploited to treat the types of cancers in comparison with standard therapeutic regimens based on overall survival (OS), progression-free survival (PFS), and time to recurrence (TTR) diagrams, and using the statistical model of Kaplan-Meier survival diagram. For the achievement and logical analysis of statistical relationships, the parameter of Incremental Cost-Effectiveness Ratio (ICER) was employed.

Besides, statistical study models having a precision medicine approach, i.e., bucket trial and umbrella trial, with high heterogeneity of population distribution were exploited due to the small statistical population of cancer patients, particularly advanced types with the characteristic of mismatch repair deficiency. It is worth mentioning that the population of patients who are included in the basket study must have a specific biological marker, and this specific marker must have a common characteristic among all types of cancer tumors. Thus, the cohort population in these trials consists of cancer patients who have a substantial spread in histology, clinical presentations, and various organ involvements. Therefore, a high heterogeneity will have existed in the study population, which results in taking into account other considerations such as the choice of the comparison arm and the selection of the type of outcome to be assessed.

Moreover, the high heterogeneity and difference in the patients investigated in these studies with different types of cancer causes changes in the outcomes and should be taken into consideration in its measurement. Among the types of techniques applied in the evaluation of the outcome or put simply the effectiveness of interventions in basket-type trials is the analysis of subgroups. Nevertheless, in cases where genetic changes cause rare types of cancers, the population experiencing a particular type of tumor that has a biological marker is limited. So, regarding the inadequate population share from a statistical perspective in any of the subgroups, the corresponding analysis will not have the correct statistical power for evaluating the effectiveness of the intervention. Furthermore, carrying out a basket clinical trial intends to generally gain the effectiveness of an intervention in all types of tumors with a common biomarker. Therefore, attempts are made to reach and aggregate the results of different subgroups based on the type of tumor. Thus, the clinical outcomes achieved in the basket study will be associated with the whole population having a common biomarker with different types of diseases. For considering the weight of all factors in the algorithms, two categories of major and minor variables were intended in the claimed invention to take into account the differences and sources of population heterogeneity.

The major variables are as follows:
1—Biomarker (FBM): Gene-specific biomarker.
2—Clinical Presentations (FCP): They include characteristics such as disease severity and disease history.
3—Health Utility (FHU): The health utility can be influenced by behaviors, beliefs, risk tolerance, and preferences in the selection of a treatment model in patients.
4—Demographics (FD): Demographics include age, sex, and income characteristics.

The minor variables are as follows:
1—Uncertain Effects of Treatment (FUET): It refers to the unknown physiological, psychological, and genetic characteristics in people that can influence the effects that patients receive from the intervention; e.g., the characteristics that can affect the patients' adherence to treatment, and these factors themselves will have an influence on the outcome of the desired intervention.
2—Adverse drug reactions (FADR): The most critical factor in creating censored data during the treatment process is because of the failure to complete the treatment path.

It is worth noting that the most influential contribution to determining and predicting the optimal cost-effectiveness of treatment approaches in the precision medicine approach is dependent on certain percentages of the mentioned major variables, and the weight of minor variables can be overlooked. Moreover, the statistical method of non-dependent two-stage analysis has been exploited in order to prevent the creation of false positive results.

As previously discussed, in the claimed invention, the parameter related to the incremental cost-effectiveness ratio (ICER) has a vital role as a determining factor for the optimal path of treatment. On the basis of its concept, the latter parameter has the following relation:

$$ICER = \frac{Δ cost ( cost of {R}_{1} - cost of {R}_{2} )}{ΔEffect ( effect of {R}_{1} - effect of 𝔱he {R}_{2} )} \qquad [ Math . ]$$

In this equation, the variable R1 is the first treatment approach (which in the claimed invention is the targeted treatment approach based on precision medicine) and R2 is the second treatment approach (which in the claimed invention is the standard treatment approach used (i.e., standard of care). To make the path of analysis and modeling smoother, the RICER parameter is defined in the following way:

$$RICER = \frac{Δ Effect ( effect of {R}_{1} - effect of 𝔱he {R}_{2} )}{Δcost ( cost of {R}_{1} - cost of {R}_{2} )} \qquad [ Math . ]$$

To carry out statistical analysis, the results of three diagrams of overall survival (OS), progression-free survival (PFS), and complete-response survival (CRS) with the Kaplan-Meier approach are exploited. In this way, the results of the performed clinical trials and observations, and consequently, the claimed Kaplan-Meier plots gained are utilized for extracting algorithms and parametric statistical relationships. Actually, a set of parametric statistical relations and equations, which are mentioned in the following, is achieved by taking into account the major variables and extracting their statistical values in the statistical populations under study. Initially, in order to compare the effectiveness of the intended treatment approach in precision medicine, with the standard comparison arm, the calculation of the area under the curve (AUC) is applied by considering the amount obtained as equivalent to the weighted average of the effectiveness, in accordance with the following equation:

$$A U C = {\sum }_{i = 1}^{n} ( {\prod }_{i : {t}_{i} \leq 𝔱}^{i} ( 1 - \frac{{d}_{i}}{{m}_{i}} ) \times ( {t}_{i + 1} - {t}_{i} ) ) \qquad [ Math . ]$$

In this general equation, di indicates the number of people who have suffered a specific event at time tᵢ, and nᵢ represents the number of people who have not suffered an event or change in the direction of data censoring (e.g., this event is death in the OS diagram, and this event is death or worsening of the disease and exit from the progressive-free state in the PFS diagram)

Regarding the multiple parameters composing any of the claimed major variables, the following matrices can be actually written for each of the listed variables in a general way to fulfill a logical algorithm.

$${ψ}_{FBM} = ( \begin{matrix}f ( {GM}_{1} ) \\ f ( {GM}_{2} ) \\ f ( {GM}_{3} ) \\ ⋮ \\ ⋮ \\ f ( {GM}_{n} )\end{matrix} ) \cdot ( \begin{matrix}w ( {GM}_{1} ) & w ( {GM}_{2} ) & w ( {GM}_{3} ) & … & ⋯ & w ( {GM}_{n} ) )\end{matrix} \qquad [ Math . ]$$

$${ψ}_{FD} = ( \begin{matrix}f ( age ) \\ f ( sex ) \\ f ( HH )\end{matrix} ) \cdot \begin{matrix}w ( age ) & w ( sex ) & w ( HH ) )\end{matrix} \qquad [ Math . ]$$

$$( \begin{matrix}f ( {comorbidity}_{1} ) \\ f ( {comorbidity}_{2} ) \\ f ( {comorbidity}_{3} ) \\ ⋮ \\ ⋮ \\ f ( stage tumor )\end{matrix} ) \cdot ( \begin{matrix}w ( {comorbidity}_{1} ) & w ( {comorbidity}_{2} ) & w ( {comorbidity}_{3} ) & … & ⋯ & w ( stage tumor ) )\end{matrix} \qquad [ Math . ]$$

In the claimed relations, the major variable of the gene-specific biomarker with FBM_{ψ}, each gene marker with GM, and the function related to the presence or absence of the gene marker and the weight of its effect in the main function associated with the major variable with f(GM) and w(GM) are represented, respectively. The major variable of demographics with FD_{ψ}, the functions related to age, sex, type of history of effective habits such as smoking, drug use, chronic drug use, lifestyle, and regime style, as well as stage of the tumor, are shown with the abbreviations f(age), f(sex)), f(HH), and f(TNM), respectively, and the weight of the effect of each in the main function related to the major variable are indicated with w(age), w(sex), w(HH), and w(TNM), respectively. The major variable of clinical presentations is shown with the abbreviation FCP_{ψ}, and the functions related to the type of diseases associated with cancer and the weight of each effect in the major variable are illustrated with f(comorbidity) and w(comorbidity), respectively.

By referring to the dimensionless nature of the claimed major variables and applying the linear algebraic summation method, the general equation of the cost-effectiveness prediction algorithm claimed in this invention is gained in the form of the following equation:

$$Effect ≡ {Ψ}_{effectiveness predictor} = a {ψ}_{FBM} + b {ψ}_{FD} + c {ψ}_{FCP} \qquad [ Math . ]$$

Concerning the multiplicity of parameters affecting each of the major variables claimed, a large statistical population is required for extracting the numerical coefficients of the functions associated with any of the claimed major variables. With respect to the statistical population size existing in the scale of precision medicine, rather than using the recent relation, or referring to the results achieved by Kaplan-Meier plots, the following statistical relations are used.

Initially, the classification of the results and the drawing of overall survival, progressive-free survival, and complete response survival diagrams with the Kaplan-Meier model are conducted in clinical trials on the basis of the presence of each biomarker, taking into account that the major weight constituting biomarkers of cancer severity and the mismatch repair deficiency model in each patient independently is, one or at most, a combination of the presence of two gene-specific markers, specifically for the same patient.

Next, the value of AUC in all three diagrams specific to the presence of each gene biomarker is computed. By calculating the AUC ratio of the same diagrams for gene-specific biomarkers in both target therapeutic regimen approaches, i.e., the precision medicine approach as the main arm and the standard of care as the comparison arm, it is realized that the primary gene-specific biomarker of the target therapeutic regimen with precision medicine approach has the most effectiveness, and it is intended as a genetic biomarker in the prediction algorithm. To analyze the achieved results, it is worth mentioning that two modes in the study population share in clinical trials will be feasible for analyzing and drawing overall survival, progressive-free survival, and complete response survival diagrams with the Kaplan-Meier model.
First, a favorable or almost favorable statistical population for the analysis and drawing of the claimed diagrams in the relevant trials should be investigated.
Second, a favorable statistical population size for the analysis and drawing of the claimed diagrams in clinical trials should not be examined or is not available for the relevant study.

Initially, the first case is evaluated.

The relations associated with the items claimed are as follows:

$${ψ}_{1 i} = \frac{\frac{{AUC}_{{GM}_{i} / OS - {R}_{1}}}{{AUC}_{{GM}_{i} / OS - {R}_{2}}}}{{\sum }_{i = 1}^{n} ( \frac{{AUC}_{{GM}_{i} / OS - {R}_{1}}}{{AUC}_{{GM}_{i} / OS - {R}_{2}}} )} \qquad [ Math . ]$$

$${ψ}_{2 i} = \frac{\frac{{AUC}_{{GM}_{i} / PFS - {R}_{1}}}{{AUC}_{{GM}_{i} / PFS - {R}_{2}}}}{{\sum }_{i = 1}^{n} ( \frac{{AUC}_{{GM}_{i} / PFS - {R}_{1}}}{{AUC}_{{GM}_{i} / PFS - {R}_{2}}} )} \qquad [ Math . ]$$

$${ψ}_{3 i} = \frac{\frac{{AUC}_{{GM}_{i} / CRS - {R}_{1}}}{{AUC}_{{GM}_{i} / CRS - {R}_{2}}}}{{\sum }_{i = 1}^{n} ( \frac{{AUC}_{{GM}_{i} / CRS - {R}_{1}}}{{AUC}_{{GM}_{i} / CRS - {R}_{2}}} )} \qquad [ Math . ]$$

$$a {ψ}_{FBM} = α {ψ}_{1 i} + {βψ}_{2 i} \qquad [ Math . ]$$

In these equations, the area under the curve is represented with AUC, the intended gene marker with GMi, the main target treatment arm with the precision medicine approach with R1, and the comparison arm or the standard of care method with R2, respectively. To specify the amount of alpha coefficient and afterward, beta coefficient, which denotes the weight of any function in the major variable function of biomarker, abbreviated as FBM_{ψ}, the following two relations are utilized:

$$α + β = 1 \qquad [ Math . ]$$

$$β = \frac{{AUC}_{G M / PFS - {R}_{2}}}{{AUC}_{GM / OS - {R}_{2}}} \qquad [ Math . ]$$

In this relation, the area under the curve is shown with AUC, the intended gene marker with GMi, and the comparison arm or the standard of care method with R2, respectively. Thus, the equation related to the major variable function of biomarker, FBM_{ψ}, and its weight in the main function of effectiveness prediction will be as follows:

$$a {ψ}_{FBM} = ( 1 - \frac{{AUC}_{GM / PFS - {R}_{2}}}{{AUC}_{GM / OS - {R}_{2}}} ) \times \frac{\frac{{AUC}_{{GM}_{i} / OS - {R}_{1}}}{{AUC}_{{GM}_{i} / OS - {R}_{2}}}}{{\sum }_{i = 1}^{n} ( \frac{{AUC}_{{GM}_{i} / OS - {R}_{1}}}{{AUC}_{{GM}_{i} / OS - {R}_{2}}} )} + ( \frac{{AUC}_{G M / PFS - {R}_{2}}}{{AUC}_{GM / OS - {R}_{2}}} ) \times \frac{\frac{{AUC}_{{GM}_{i} / PFS - {R}_{1}}}{{AUC}_{{GM}_{i} / PFS - {R}_{2}}}}{{\sum }_{i = 1}^{n} ( \frac{{AUC}_{{GM}_{i} / PFS - {R}_{1}}}{{AUC}_{{GM}_{i} / PFS - {R}_{2}}} )} \qquad [ Math . ]$$

To determine the relation associated with the function and the weight of the major variable of demographics, bψ_{FD}, along with the weight of the major variable of clinical presentations, cψ_{FCP}, the following equation is exploited:

$$b {ψ}_{F D} + c {ψ}_{F C P} = γ {ψ}_{3 i} \qquad [ Math . ]$$

To determine the gamma coefficient, representing the weight of the 3iψ function in the major variable function of the demographics, analyzing the statistical results of the clinical trials of the main targeted treatment arm with a precision medicine approach is applied in the following manner:

$${γ}_{f ( age )} = 1 ; if age ∈ {Mean}_{age} ∓ {t}_{0.95} \frac{SD}{\sqrt{{N}_{CR}}} \qquad [ Math . ]$$

$$\begin{matrix}{γ}_{f ( female )} = 0.5 \times \\ ( \begin{matrix}\frac{{N}_{female} - {t}_{0.95} \frac{SD}{\sqrt{{N}_{CR}}}}{{N}_{CR} - {t}_{0.95} \frac{SD}{\sqrt{{N}_{CR}}}} + \\ \frac{{N}_{female} + {t}_{0.95} \frac{SD}{\sqrt{{N}_{CR}}}}{{N}_{CR} + {t}_{0.95} \frac{SD}{\sqrt{{N}_{CR}}}}\end{matrix} ) \& {γ}_{f ( male )} \\ = ( 1 - {γ}_{f ( female )} )\end{matrix} \qquad [ Math . ]$$

$${γ}_{f ( {HH}_{i} )} = 0.5 \times ( \frac{{N}_{{HH}_{i} - CR} - {t}_{0.95} \frac{SD}{\sqrt{{N}_{total}}}}{{N}_{HH \_ total} - {t}_{0.95} \frac{SD}{\sqrt{{N}_{total}}}} + \frac{{N}_{{HH}_{i} - CR} + {t}_{0.95} \frac{SD}{\sqrt{{N}_{total}}}}{{N}_{HH \_ total} + {t}_{0.95} \frac{SD}{\sqrt{{N}_{total}}}} ) \qquad [ Math . ]$$

$${γ}_{f ( {stage}_{i} )} = 0.5 \times ( \frac{{N}_{{stage}_{i} - CR} - {t}_{0.95} \frac{SD}{\sqrt{{N}_{total}}}}{{N}_{stage \_ total} - {t}_{0.95} \frac{SD}{\sqrt{{N}_{total}}}} + \frac{{N}_{{HH}_{i} - CR} + {t}_{0.95} \frac{SD}{\sqrt{{N}_{total}}}}{{N}_{HH \_ total} + {t}_{0.95} \frac{SD}{\sqrt{{N}_{total}}}} ) \qquad [ Math . ]$$

In these relations, the parameters of age, sex, history of personal habits, and stage of tumor in the gamma coefficient, with ageγ, sexγ, HHγ and stageγ, are respectively shown. Also, the parameters of standard deviation with SD, the total number of patients studied in the trial with Ntotal, the number of patients included in the complete response stage with NCR, the total number of patients with the target habit history with NHH-total, and the total number of patients in a specific cancer stage with NStage_total are represented, respectively.

The final value of the gamma coefficient will be gained via the following determinants:

$$γ = \vert \begin{matrix}{γ}_{f ( age )} & 0 & 0 & 0 \\ 0 & {γ}_{f ( female )} & 0 & 0 \\ 0 & 0 & {γ}_{f ( {HH}_{i} )} & 0 \\ 0 & 0 & 0 & {γ}_{f ( {stage}_{i} )}\end{matrix} \vert \qquad [ Math . ]$$

Ultimately, by placing the claimed relations, the effectiveness function and the RICER function are achieved as follows:

$$Δ Effect ≡ {Ψ}_{effectiveness predictor} = [ \begin{matrix}( 1 - \frac{{AUC}_{\frac{GM}{PFS} - {R}_{2}}}{{AUC}_{\frac{GM}{OS} - {R}_{2}}} ) \times \frac{\frac{{AUC}_{\frac{{GM}_{i}}{OS} - {R}_{1}}}{{AUC}_{\frac{{GM}_{i}}{OS} - {R}_{2}}}}{{\sum }_{i = 1}^{n} ( \frac{{AUC}_{\frac{{GM}_{i}}{OS} - {R}_{1}}}{{AUC}_{\frac{{GM}_{i}}{OS} - {R}_{2}}} )} + \\ ( \frac{{AUC}_{\frac{GM}{PFS} - {R}_{2}}}{{AUC}_{\frac{GM}{OS} - {R}_{2}}} ) \times \frac{\frac{{AUC}_{\frac{{GM}_{i}}{PFS} - {R}_{1}}}{{AUC}_{\frac{{GM}_{i}}{PFS} - {R}_{2}}}}{{\sum }_{i = 1}^{n} ( \frac{{AUC}_{\frac{{GM}_{i}}{PFS} - {R}_{1}}}{{AUC}_{\frac{{GM}_{i}}{PFS} - {R}_{2}}} )}\end{matrix} ] + \vert \begin{matrix}{γ}_{f ( age )} & 0 & 0 & 0 \\ 0 & {γ}_{f ( female )} & 0 & 0 \\ 0 & 0 & {γ}_{f ( {HH}_{i} )} & 0 \\ 0 & 0 & 0 & {γ}_{f ( {stage}_{i} )}\end{matrix} \vert \times \frac{\frac{{AUC}_{{GM}_{i} / CRS - {R}_{1}}}{{AUC}_{{GM}_{i} / CRS - {R}_{2}}}}{{\sum }_{i = 1}^{n} ( \frac{{AUC}_{{GM}_{i} / CRS - {R}_{1}}}{{AUC}_{{GM}_{i} / CRS - {R}_{2}}} )} \qquad [ Math . ]$$

$$RICER = \frac{[ \begin{matrix}( 1 - \frac{{AUC}_{\frac{GM}{PFS} - {R}_{2}}}{{AUC}_{\frac{GM}{OS} - {R}_{2}}} ) \times \frac{\frac{{AUC}_{\frac{{GM}_{i}}{OS} - {R}_{1}}}{{AUC}_{\frac{{GM}_{i}}{OS} - {R}_{2}}}}{{\sum }_{i = 1}^{n} ( \frac{{AUC}_{\frac{{GM}_{i}}{OS} - {R}_{1}}}{{AUC}_{\frac{{GM}_{i}}{OS} - {R}_{2}}} )} + \\ ( \frac{{AUC}_{\frac{GM}{PFS} - {R}_{2}}}{{AUC}_{\frac{GM}{OS} - {R}_{2}}} ) \times \frac{\frac{{AUC}_{\frac{{GM}_{i}}{PFS} - {R}_{1}}}{{AUC}_{\frac{{GM}_{i}}{PFS} - {R}_{2}}}}{{\sum }_{i = 1}^{n} ( \frac{{AUC}_{\frac{{GM}_{i}}{PFS} - {R}_{1}}}{{AUC}_{\frac{{GM}_{i}}{PFS} - {R}_{2}}} )}\end{matrix} ]}{Δ cost ( cost of {R}_{1} - cost of {R}_{2} )} + \frac{\vert \begin{matrix}{γ}_{f ( age )} & 0 & 0 & 0 \\ 0 & {γ}_{f ( female )} & 0 & 0 \\ 0 & 0 & {γ}_{f ( {HH}_{i} )} & 0 \\ 0 & 0 & 0 & {γ}_{f ( {stage}_{i} )}\end{matrix} \vert \times \frac{\frac{{AUC}_{{GM}_{i} / CRS - {R}_{1}}}{{AUC}_{{GM}_{i} / CRS - {R}_{2}}}}{{\sum }_{i = 1}^{n} ( \frac{{AUC}_{{GM}_{i} / CRS - {R}_{1}}}{{AUC}_{{GM}_{i} / CRS - {R}_{2}}} )}}{Δ cost ( cost of {R}_{1} - cost of {R}_{2} )} \qquad [ Math . ]$$

By maximizing the value of the RICER function, the most optimal cost-effectiveness will be obtained.

Now, the second state, i.e., a case where an insufficient size of the study population in the trial has been evaluated, is examined.

Regarding the above-mentioned case and concerning the primary approach of calculations and analyses performed for the maximization of the RICER function value, followed by the most optimal cost-effectiveness, and based on the major variable of biomarker, FBM v, as the central variable, the network matrix is utilized to increase the size of the examined population in the internship and consequently, more detailed statistical analysis.

In this circumstance, the major foundation at the common point of the population under study in clinical trials will be based on the presence or absence of biomarkers for different types of cancer. Taking into consideration this point of resemblance, plotting diagrams with the Kaplan-Meier model and analyzing claimed clinical results for the whole evaluated population and all cancer patients with the claimed biomarker are carried out.

It is worth noting that drawing the claimed diagrams with the Kaplan-Meier model for the standard of care method called R2 for the total number of all types of patients with different types of cancer with a common gene-specific biomarker will be based on the specific standard of care approved for each type of cancer. Subsequently, in order to specify the cost of the total average standard of care, the Cost of SoCtot, the weighted average of the population of patients with each type of cancer followed by the treatment cost of the standard of care for any type of cancer will be exploited as the following equation.

$$cost of {SoC}_{total} = {\sum }_{i = 1}^{n} {{w}_{n} ( cost of {R}_{2} )}_{n} \qquad [ Math . ]$$

In this equation, the weight number of patients for each type of cancer is represented with wn, and the intervention costs for the standard of care method for each specific cancer are determined with the Latin title cost of R2. With respect to the extensiveness of studies performed in the domain of standard-of-care procedures of cancers and consequently, lacking restrictions in the population share (the weight number of patients), it is possible to statistically take into account the uniform rate of the population tailored to the collection of examined cancers using a specific therapeutic regimen (treatment regimen) in the precision medicine approach in the claimed modeling. Hence, the cost of the total average standard of care, abbreviated Cost of SoCtot, will be simplified as follows:

$$cost of {SoC}_{total} = {\sum }_{i = 1}^{n} {( cost of {R}_{2} )}_{n} \qquad [ Math . ]$$

In this equation, the intervention costs for the standard of care method for each specific cancer are determined with the Latin title cost of R2.

Ultimately, RICER function value can be defined as follows:

$$RICER = \frac{[ \begin{matrix}( 1 - \frac{{AUC}_{GM / PFS - {R}_{2}}}{{AUC}_{GM / OS - {R}_{2}}} ) \times \frac{\frac{{AUC}_{\frac{{GM}_{i}}{OS} - {R}_{1}}}{{AUC}_{\frac{{GM}_{i}}{OS} - {R}_{2}}}}{{\sum }_{i = 1}^{n} ( \frac{{AUC}_{\frac{{GM}_{i}}{OS} - {R}_{1}}}{{AUC}_{\frac{{GM}_{i}}{OS} - {R}_{2}}} )} + \\ ( \frac{{AUC}_{\frac{GM}{PFS} - {R}_{2}}}{{AUC}_{\frac{GM}{OS} - {R}_{2}}} ) \times \frac{\frac{{AUC}_{\frac{{GM}_{i}}{PFS} - {R}_{1}}}{{AUC}_{\frac{{GM}_{i}}{PFS} - {R}_{2}}}}{{\sum }_{i = 1}^{n} ( \frac{{AUC}_{\frac{{GM}_{i}}{PFS} - {R}_{1}}}{{AUC}_{\frac{{GM}_{i}}{PFS} - {R}_{2}}} )}\end{matrix} ]}{Δ cost ( cost of {R}_{1} - cost of {R}_{2} )} + \frac{\vert \begin{matrix}{γ}_{f ( age )} & 0 & 0 & 0 \\ 0 & {γ}_{f ( female )} & 0 & 0 \\ 0 & 0 & {γ}_{f ( {HH}_{i} )} & 0 \\ 0 & 0 & 0 & {γ}_{f ( {stage}_{i} )}\end{matrix} \vert \times \frac{\frac{{AUC}_{{GM}_{i} / CRS - {R}_{1}}}{{AUC}_{{GM}_{i} / CRS - {R}_{2}}}}{{\sum }_{i = 1}^{n} ( \frac{{AUC}_{{GM}_{i} / CRS - {R}_{1}}}{{AUC}_{{GM}_{i} / CRS - {R}_{2}}} )}}{Δ cost ( cost of {R}_{1} - {\sum }_{i = 1}^{n} {( cost of {R}_{2} )}_{n} )} \qquad [ Math . ]$$

Then, an example will be rendered.

Investigating the effectiveness of Pembrolizumab monoclonal antibody drug in endometrial, stomach, and pancreatic cancers, characterized by deficient mismatch repair (MMR), with a precision medicine approach.

In the target treatment approach, as the main arm of the study, a therapeutic regimen containing 200 mg of intravenous injection every 3 weeks in contrast to the standard of care comparison arm is exploited. The population in this study consists of 49 patients with endometrial cancer, 24 patients with gastric (stomach) cancer, and 22 patients with pancreatic cancer. Moreover, the studied statistical population in the comparison arm with the standard of care regimen includes 49 patients with endometrial cancer, 29 patients with gastric (stomach) cancer, and 31 patients with pancreatic cancer.

On the basis of the claims, the major variable of the biomarker (FBM) cited in the clinical trial performed with the Pembrolizumab (humanized monoclonal antibody therapy) is the cause of mismatch repair deficiency and subsequently, with the characteristic of microsatellite instability (MSI-H/dMMR). Actually, the presence of the MSI-H/dMMR biological agent, in the histological and biological characteristics of the tumor, will be a priority factor in the cost-effectiveness prediction algorithm, taking into account the effectiveness function in the RICER main function, to specify the therapeutic regimen containing the claimed drug.

Now, to assess the claimed prediction algorithm, we return to the claimed relation for it with the title of the RICER function

$$RICER = \frac{[ \begin{matrix}( 1 - \frac{{AUC}_{\frac{GM}{PFS} - {R}_{2}}}{{AUC}_{\frac{GM}{OS} - {R}_{2}}} ) \times \frac{\frac{{AUC}_{\frac{{GM}_{i}}{OS} - {R}_{1}}}{{AUC}_{\frac{{GM}_{i}}{OS} - {R}_{2}}}}{{\sum }_{i = 1}^{n} ( \frac{{AUC}_{\frac{{GM}_{i}}{OS} - {R}_{1}}}{{AUC}_{\frac{{GM}_{i}}{OS} - {R}_{2}}} )} + \\ ( \frac{{AUC}_{\frac{GM}{PFS} - {R}_{2}}}{{AUC}_{\frac{GM}{OS} - {R}_{2}}} ) \times \frac{\frac{{AUC}_{\frac{{GM}_{i}}{PFS} - {R}_{1}}}{{AUC}_{\frac{{GM}_{i}}{PFS} - {R}_{2}}}}{{\sum }_{i = 1}^{n} ( \frac{{AUC}_{\frac{{GM}_{i}}{PFS} - {R}_{1}}}{{AUC}_{\frac{{GM}_{i}}{PFS} - {R}_{2}}} )}\end{matrix} ]}{Δ cost ( cost of {R}_{1} - cost of {R}_{2} )} + \frac{\vert \begin{matrix}{γ}_{f ( age )} & 0 & 0 & 0 \\ 0 & {γ}_{f ( female )} & 0 & 0 \\ 0 & 0 & {γ}_{f ( {HH}_{i} )} & 0 \\ 0 & 0 & 0 & {γ}_{f ( TNM {stage}_{i} )}\end{matrix} \vert \times \frac{\frac{{AUC}_{{GM}_{i} / CRS - {R}_{1}}}{{AUC}_{{GM}_{i} / CRS - {R}_{2}}}}{{\sum }_{i = 1}^{n} ( \frac{{AUC}_{{GM}_{i} / CRS - {R}_{1}}}{{AUC}_{{GM}_{i} / CRS - {R}_{2}}} )}}{Δ cost ( cost of {R}_{1} - cost of {R}_{2} )}$$

For the calculation, the area under the curve (AUC) values of OS, PFS, and CRS diagrams of both intended therapeutic regimens with the precision medicine approach and the comparison arm with the standard of care approach are needed. Concerning the statistical results and the Kaplan-Meier diagrams, the values of the area under the curve of these two therapeutic regimen approaches for endometrial cancer, as an example of the three cancers investigated in this clinical trial, are as follows:

$${AUC}_{\frac{{MSI}_{H} / dMMR}{OS} - Pembrolizumab} = 15.89 \qquad [ Math . ]$$

$${AUC}_{\frac{{MSI}_{H} / dMMR}{OS} - SOC} = 14.16 \qquad [ Math . ]$$

$${AUC}_{\frac{{MSI}_{H} / dMMR}{PFS} - Pembrolizumab} = 12. 65 \qquad [ Math . ]$$

$${AUC}_{\frac{{MSI}_{H} / dMMR}{PFS} - SOC} = 10.87 \qquad [ Math . ]$$

$${AUC}_{\frac{{MSI}_{H} / dMMR}{CRS} - Pembrolizumab} = 9.89 \qquad [ Math . ]$$

$$AU {C}_{\frac{{MSI}_{H} / dMMR}{C R S} - SOC} = 2.94 \qquad [ Math . ]$$

In recent relations, the comparison arm with the standard of care (SOC) approach is illustrated.

To examine the value of the area under the curve of the CRS diagram and subsequently, to determine the constituent parameters of the gamma coefficient in the major variable part of the demographics in the effectiveness function, the statistical results for the parameters of age range, the share of both sexes, and the stage of tumor are achieved as follows:

$${γ}_{f ( age )} = 1 ; if age ∈ 31 ( 26. 8 , 35 \text{.2} ) \qquad [ Math . ]$$

$${γ}_{f ( female )} = 0.68 \& {γ}_{f ( male )} = 0.32 \qquad [ Math . ]$$

$${γ}_{f ( {HH}_{i} )} = 1 \qquad [ Math . ]$$

$${γ}_{f ( {stage}_{1} )} = 0.228 \qquad [ Math . ]$$

$${γ}_{f ( {stage}_{1} )} = 0.3 \; 21 \qquad [ Math . ]$$

$${γ}_{f ( {stage}_{1} )} = 0.249 \qquad [ Math . ]$$

$${γ}_{f ( {stage}_{4} )} = 0.212 \qquad [ Math . ]$$

Using the coefficients gained from the recent trial and the claimed determinants, the weight of the effect of demographics in the claimed effectiveness function can be calculated.

In summary, the choice of a plausible effective therapeutic regimen for a patient suffering from cancer is one of the most critical issues in hospitalized patients due to its high potential for being mostly ineffective in the long-term complications in the patient's treatment path. On the one hand, this matter has led to the disease progression and declined the patients' quality of life because of lacking sufficient effectiveness, and on the other hand, it has caused numerous other problems in the patient's quality of life due to the extensive range of complications, particularly in the long-term use of the relevant regimen. Subsequently, these cases not only enhance but also cause a sharp decrease in the QALY (Quality-adjusted life-years) and DALY (disability-adjusted life-years) scales in patients. As well as, another important thing is the heavy costs of treating patients. Aligned with the low effectiveness of the therapeutic regimen followed by the reduction of the QALY and DALY scale in the vast majority of cases, it practically incurs ineffectual and unreasonable expenses on the health system of the countries, the family of the patient affected by the disease, and even the patient himself/herself. Nowadays, precision medicine could acceptably diminish the problem of treatment effectiveness and result in increasing the QALY and DALY scales and even lead to the complete recovery of patients; however, this improvement relies on an exorbitant expense, which is out of the ability of the treatment system of all countries and the vast majority of patients and their families. Thus, the claimed invention employed an algorithm for predicting the cost-effectiveness of novel therapeutic regimen, which provides the possibility for the treatment system and oncology physicians to choose the most optimal method of treatment from the perspective of the maximum effectiveness with the minimum amount of cost with a precision medicine approach, through this cost-effectiveness forecasting algorithm, before beginning the treatment period of patients by referring to biomarker (FBM), demographics (FD), and clinical presentations (FCP) of patients. This will greatly contribute both to optimizing the amount of increasing the QALY and DALY scales of the patient, elevating the morale and compliance of the patients in the continuation of the treatment process, and enhancing the confidence of the treatment system.

Notably, the industrial application of the claimed invention is in the domain of Health Economic and Outcome Research (HEOR).

1. In the claimed invention, a multivariable cost-effectiveness prediction algorithm is employed for the choice of the type of interventions, therapeutic approach, and therapeutic regimen in severe, chronic, and life-threatening diseases, such as cancer, designed with a precision medicine approach.

2. According to claim 1, the claimed algorithm is applicable to advanced diseases with progressive, life-threatening characteristics, limited statistical population, heavy, expensive, chronic therapeutic regimens with low effectiveness and low probability to reach the complete response phase (full recovery), such as malignancies and cancerous tumors.

3. According to claim 1, the claimed algorithm is composed of two general parts: effectiveness function and treatment cost function.

4. According to claim 3, the main function of the algorithm is specified by referring to the inverse value of the ICER factor, called RICER in the claimed invention, and it consists of the ratio of the claimed effectiveness function to the treatment cost function

5. According to claim 3, the effectiveness function is a multivariate function depending on clinical presentations and genetic characteristics and the degree of tumor-malignancy involvement, which relies on three major variables (with a major weight) for modeling, analyzing, and comparing different therapeutic approaches and selecting the treatment method.

6. According to claim 5, analyzing the results of statistical studies and clinical trials performed for various approaches and different therapeutic regimens are utilized to determine the amount and weight of the claimed variables in the effectiveness function.

7. According to claim 6, the preferred model of studies and clinical trials claimed in patients who experienced the claimed diseases is the basket trial model, taking into account the precision medicine approach.

8. According to claim 6, the Kaplan-Meier model, which is a kind of statistical survival analysis approach, is applied to specify the amount and weight of the claimed variables.

9. According to claim 6, the claimed major variables in the claimed effectiveness function embrace biomarker, demographics, and clinical presentation.

10. According to claim 9, the variable biomarker includes the presence or absence of a genetic factor, a significant increase or decrease of a pre-existing genetic factor, or a specific feature of cancer tumor cells, such as the presence of different mechanisms in creating drug resistance or advanced patterns of gene mutations, like mismatch repair deficiency.

11. According to claim 10, in the process of designing the claimed algorithm, based on the centrality of only one biomarker in the patients involved in the alleged diseases, the statistical population is classified, studied, and modeled from the claimed patterns on the basis of the presence or absence of the gene biomarker.

12. According to claim 10, the primary weight constituting the major variable of demographics in the claimed effectiveness function is taken into account, relying on three parameters, including the type of sex (physiological), age range, and specific habit history of in the patient's lifestyle.

13. To assess and compare the effectiveness ratio of the intended regimen with the standard of care regimen, the overall survival (OS), progression-free survival (PFS), and complete-response survival (CRS) diagrams are exploited, referring to the survival analysis approach.

14. According to claim 13, to compare the effectiveness of the target therapeutic regimen with the standard of care approach, the ratio of the area under the curve (AUC) of the claimed diagrams is used, taking into consideration the clinical results gained from two statistical population of the trial, one by the target therapeutic approach and the other by the standard of care approach.

15. According to claim 14, the major variable of the claimed biomarker is specified by achieving the maximum ratio of the area under the curve (AUC) of OS and PFS diagrams of the target therapeutic approach relative to the standard of care approach.

16. According to claim 15, the major variable of the claimed biomarker is dependent on two variables, which will be, respectively, the ratio of the area under the curve of the OS and PFS diagrams of the biomarker selected separately to the total area under the curve of the OS and PFS diagrams of total biomarkers contributing and effective in changing the effectiveness of therapeutic regimens, in both approaches, i.e., the intended therapeutic regimen and standard of care approach with specific weight.

17. According to claim 16, the claimed weights will be obtained from the ratio of the area under the curve of the OS diagram to the area under the curve of the PFS diagram in the target therapeutic regimen approach for the dependent variable to the ratio of the area under the curve of the claimed comparison PFS diagram for the two claimed therapeutic approaches, and also from 1 minus the value of the ratio of the AUC of the OS diagram to the AUC of the PFS diagram in the target therapeutic regimen approach for the dependent variable to the ratio of the AUC of the claimed comparison PFS diagram for the two claimed therapeutic approaches.

18. According to claim 17, the major variable of the claimed demographics depends on the ratio of the area under the curve of the CRS diagram of two claimed therapeutic approaches in the presence of the claimed biomarker selected to the total area under the curve of the CRS diagram of the two claimed therapeutic regimen approaches in the total biomarkers contributing and effective in changing the effectiveness of therapeutic regimens with specific weight.

19. According to claim 18, the weight constituting the major variable of clinical presentations in the claimed effectiveness function is intended to be dependent on the parameter of patients' stage of tumor and the condition of their being metastatic or not.

20. According to claim 19, the weight of the considered function, the two major variables of demographics and clinical presentations in the claimed effectiveness function, will be computed from the determinants of the specific weight coefficients for the four claimed parameters, i.e. type of sex, age range, specific habit history in lifestyle, and patient's stage of the tumor.

21. According to claim 20, to determine the weight coefficient of the claimed type of sex parameter, the ratio of the weight of both sexes in the clinical trial conducted for the target therapeutic approach compliance with the precision medicine, which enrolled the complete response phase of treatment, has been exploited in the claimed complete-response survival (CRS) approach.

22. According to claim 20, to determine the weight coefficient of the claimed age range parameter, the ratio of zero to one, to place the age range of each patient in the optimal age range of the studied patient population, in the clinical trial carried out for the target therapeutic approach according to precision medicine, who have entered the complete treatment response phase in the claimed complete-response survival (CRS) approach has been used. Actually, being in the claimed age range means better response, effectiveness, and higher utility of treatment for these patients relative to patients who are outside the claimed age range.

23. According to claim 20, to determine the weight coefficient of the claimed specific habit history in patients' lifestyle parameter (HH), the ratio of zero to one has been used for the presence or absence of that habit intended for each patient to the habit considered for the total number of studied patients in the clinical trial performed for the target therapeutic approach according to precision medicine, who have entered the complete treatment response phase in the claimed complete-response survival (CRS) approach. Indeed, the existence of the claimed habit history means better response, effectiveness, and higher utility of treatment for these patients compared to patients who are out of the claimed habit history. Among these histories of habits in lifestyle are smoking, drug abuse, and chronic taking of medicines.

24. According to claim 20, to determine the weight coefficient of tumor stage parameter, the ratio of effectiveness for the population of patients studied in both therapeutic approaches, with the ratio of the effectiveness of the target therapeutic approach according to precision medicine, to the effectiveness of the standard of care method, who have entered the phase of complete treatment response, with the claimed complete-response survival (CRS) approach has been utilized.

25. In the case of a small statistical population in the clinical trials conducted to check the effectiveness of the target drug with precision medicine approach, to increase the population, integrating and expanding the results and clinical data of various trials carried out by the claimed drug, in different cancers, with the commonality in connection with the presence of the claimed biomarker and subsequently, the similarity in the amount and effect of this claimed variable in the effectiveness of the treatment will be employed.

26. According to claim 25, to compare the clinical results of the effectiveness ratio of the target therapeutic regimen with the standard of care regimen, on the basis of the survival analysis approach from the claimed diagrams, the integration and the expansion of the clinical and statistical results gained in the clinical trials conducted with the standard of care regimen in each cancer will be exploited in a total standard of care (SoCtotal) regimen, taking into account the existence of the claimed commonality.

27. According to claim 26, the method for integrating clinical results, with the network matrix method and integrating all the data and results of clinical internships and consequently, the OS, PFS, and CRS diagrams will be based on claimed Kaplan-Meier model and drawing an OS diagram, a PFS diagram, and a general CRS diagram.
